(19) United States
(12) Patent Application Publication
(10) Pub. No.: US 2004/0146885 A1
(43) Pub. Date: Jul. 29, 2004

(54) Multiplexing array techniques

(76) Inventor: Nathaniel Tue Tran, Irvine, CA (US)

Correspondence Address: Nathaniel Tue Tran, 3205 Aspen Grove, Irvine, CA, 92618-4085, US

(21) Appl. No.: 10/667,882

(22) Filed: Sep. 22, 2003

Related U.S. Application Data
(60) Provisional application No. 60/443,017, filed on Jan. 28, 2003.

(51) Int. Cl. C12Q 1/68; G01N 33/53

(52) U.S. Cl. 435/6; 435/7.1

(57) ABSTRACT

A novel method for labeling, detecting and quantifying molecules from multiple samples on the same array is described. The method uses at least one labeled sample to be mixed together with an unlabeled sample and allows competitive binding to an array. The array profiles various molecules from both samples at predetermined locations for detection and quantitative comparison. The signals detected for various molecules from labeled sample will provide indication of presence and relative amount of the same molecules in the unlabeled sample.

### CROSS-REFERENCE TO RELATED APPLICATION

This application claims priority of provisional application U.S. Serial No. 60/443,017, filed Jan. 28, 2003, the content of which is incorporated herein by reference.

### BACKGROUND OF THE INVENTION

Since the completion of the human genome project, the emphasis on protein analysis has led to a new field of science called “proteomics”. The currently well-adopted proteomic analysis method involves: (1) labeling proteins from two or more sources with different colored fluorescent dyes, (2) then separating them on 2 dimensional gels, (3) looking for proteins level changes in different dots of proteins base on differently colored fluorescent intensity, and finally (4) the proteins in the dots of interest can be cut out and identified using mass spectrometry. DNA microarray multiplexing of samples can be performed using similar fluorescent dyes to differentially display thousands of expressed genes (in the form of RNAs). Unfortunately, the same use of fluorescent dyes on protein microarrays has suffered drawback such as denatured proteins, destroyed or altered binding due to fluorescent dyes that are too bulky. All because unlike DNA or RNA, proteins are much more fragile and require delicate 3-dimensional shapes to function properly.

Recently there has been an improved method that allows multiplexing of two samples on the same protein array. This method uses heavy stable isotope labeling such as ¹⁵N incorporation into proteins as they are synthesized. The proteins with heavy isotope and normal proteins are mixed together and applied to the same array. Proteins bound to each spot are then analyzed with highly sensitive mass spectrometry. Because proteins from ¹⁵N labeling source are slightly heavier, they result in separate (shifted) peaks in the mass spectra allowing quantitative comparison with proteins from unlabeled source. A variation of this method uses isotope-coded affinity tag (ICAT) labeling reagents to label proteins that cannot be labeled by metabolic incorporation. The main drawback of this method is that if only 1% of the protein's abundance changes then mass spectrometry have to perform 99% of redundant work to identify this 1%. This drawback significantly reduces the high-throughput capability and calls for a better method that can enable quantitative comparison directly on the array.

One of the benefits that has arisen, from the advent of the array, is that multiple mini assays can be performed simultaneously using minute amount of test samples and reagents. While 2-D gel electrophoresis and mass spectrometry are time consuming, these technologies are capable of displaying the changes in many proteins expression and identify the proteins themselves. Two identical protein arrays can be used to captured proteins from two sources for qualitative comparison. With more advance technology, one protein array can be used to capture proteins from two samples for quantitative comparison. For example, one sample may be derived from cells grown in a normal medium, while the other sample is derived from cells grown in heavier isotope containing medium. Using mass spectrometry to identify the protein also allows quantitative comparison between two or more samples because of the slight difference in mass between the same proteins in different samples due to the introduction of heavier isotopes. This method thus allows quantitative multiplexing of protein samples on protein arrays, but still requires highly advanced mass spectrometry, and still is very time consuming.

Arrays, Biochips

A flat surface with group of molecules spotted on equidistant from each other form a matrix called a biochip or array (hereinafter called an “array”). An array usually comprises binding agents to bind to molecules to be analyzed from a sample. The binding agents capture and display the specific molecules at a specific location on the array for qualitative and quantitative analyses. The main purpose of the array is to combine and miniaturize many assays so that multiple assays can be done simultaneously with only a small and singular amount of sample needed for analysis.

Most commonly, an array is used to detect particular interaction like DNA/DNA or DNA/RNA hybridization, antigen/antibody pairing, protein/protein interaction, and ligand/receptor binding. However, an array can be used to identify, quantify, and study the affinity, or other reactions, of any group of molecules toward another, either directly or in conjugation with one another.

An antibody array is made of solid support comprising a plurality of monoclonal antibodies or binding fragments of monoclonal antibodies derived from one or more animals or organisms having unique specificity for one or more antigens affixed to the solid support at a non-binding region of the antibody or fragment, leaving a binding region available to bind one or more antigens upon contact. Thereon, a plurality of antibodies comprises binding specificities for a plurality of antigen. Such an array can comprise, for example, a plurality of: monoclonal antibodies, binding fragments of monoclonal antibodies, proteins that are known to specifically interact with other proteins, aptamers, and nucleotide sequences that specifically recognize and bind to specific protein. For simplicity, an antibody referred to herein will also include all other type of molecules and binding agents that would recognize and bind specifically to other molecules called antigens for simplicity.

For simplicity, an array will be discussed in terms of an array wherein the resident molecule is an antibody or binding agent and the potentially binding or reacting molecules include potential antigens. When an array of immobilized antibodies is exposed to one or a number of potential antigens, the portion of the array will comprise antibody-antigen bound pairs. The bound pairs depend upon the concentration of the antigen and the affinity of the antibody for the antigen. This binding allows hundreds of antigen to be profiled on an array at once. An array, however, may be used to similarly study the interaction of any two or more molecules. Such interaction arrays use technology such as surface plasmon resonance to determine the binding affinity between the resident molecules and the sample's molecules.

Commonly, a test solution of unknown antigen concentration is compared with a series of standard solutions of known concentrations of known antigen to inhibit competitively the binding of the unknown antigen to the antibody. Such assays are used to determine the most effective antigen.

International patent number WO 239,120 discloses the use of antibody arrays for detecting various known and unknown proteins that are present in diseased and normal cells. By using identical arrays to capture proteins present in a disease and normal tissue, one can determine the presence or absence of each protein in each of the tissue by detecting the labeled proteins bound to the antibody spots. Identifying the presence of each protein allows one to determine the potential role of each protein in the disease process. Such proteins can be potential targets for therapeutic intervention or can potentially be used as markers for diagnosis of diseases.

Examples of common arrays comprise a plurality of monoclonal antibodies with known specificities for other molecules, or other binding agents like protein A, protein G (a binding agent for IgG), antigenic epitopes (to facilitate the detection of specific antibodies i.e. anti Myelin Basic Protein present in serum indicate Multiple Sclerosis, anti nuclear antibody presented in Systemic Lupus Erythematosus), jack fruit extract (a binding agent for IgA), and bacterial toxoids or proteins with known specific interaction to other bio-molecules small molecules like avidin and biotin, specific DNA domains that only certain proteins interact with.

Currently, micro arrays are reaching ever-smaller proportions and are often referred to as “chips”, although their dimensions can vary. Arrays of smaller dimensions can conveniently provide quick and convenient sample, or subset of samples, analysis. For purposes in this document, macro and micro arrays will be referred to collectively as “arrays”.

Protein arrays are made by spotting proteins onto supported Polyvinyl-diflouride (PVDF) membranes or slides with the use of commercially available slide-spotters AKA arrayers. PVDF membranes will adsorb protein and retain it well enough for the purpose of the assay. The major disadvantage is that this method can presently only produce a macroarrray having low density. The membrane can be supported by a sturdy plastic backing to keep it from bending and warping making detection and quantification easier.

To achieve higher density, proteins need to be covalently coupled to a sturdy surface such as plastic or glass. An example of such coupling procedure was described in Houseman et. al. Nature Biotechnology 20:270. Multiple spots of the same monoclonal antibody can be used to improve reliability of the analysis. Spots of monoclonal antibodies against molecules known to be present equally among the samples are used for internal calibration standard. The array is then subjected to treatments that will block other nonspecific attachment. For PVDF membranes, for example, a typical procedure is to saturate the membrane with non- specific proteins by incubating with non-fat milk, or bovine serum albumin solution. Each array only needs to carry a set amount of spots necessary to carry out certain analyses or medical diagnoses.

DNA arrays are similarly made but only by covalently link DNA molecules to a solid support. The DNA fragments uses are single stranded DNA derive from natural or synthetic sources. Typically, the solid surface is preactivated to react and form covalent bonds with any biological molecules spotted onto it. DNA are then spotted on and the remainder of the surface deactivated to prevent further DNA capturing.

Quantification

Antibodies are used frequently to quantify different antigens in medical laboratory testing most notably in Enzyme Linked Immuno Adsorbance Assay (ELISA). These tests are among the most expensive and time consuming of all. Currently, physicians have to take samples from patients and send off for lab testing. Only laboratories with dedicated equipments and the appropriate certification can perform these tests. This process is not only laborious and time consuming, but also costly and inefficient. Sometimes, the patient's life requires that the physician in charge make a quick decision, which requires the test results. This invention seeks to place such lab testing in the physician's office using the lab-on-a-chip concept. The equipments and processes involve can be fully automated making in-office lab testing quick, efficient, and reliable.

Many of the modern immuno-chemical methods of quantification are based upon having a simple and accurate method to measure the quantity of indicator molecules. When the indicator molecule is labeled with a radioisotope, it may be quantified by counting radioactive decay events in a scintillation counter. This assay is called a radioimmunoassay or RIA. For an enzyme-linked immunoadsorbent assay or ELISA, the indicator molecule is covalently coupled to an enzyme and may be quantified by determining with a spectrophotometer the initial rate at which the enzyme converts a clear substrate into a colored product.

The disadvantage of present protein arrays is that the methods of detection and quantification for comparison involve comparing the signals on two identical but distinct arrays exposed to two different sets of antigens. The first major problem is that if the amount of a particular antigen over-saturate the amount of the reacting antibody, i.e., there is more of that particular antigen than there is enough antibody to bind, then that particular spot is useless for the purpose of quantification or even quantitative comparison. In addition even if the same set of antigen is exposed to the two identical arrays for experimental purpose, it's unlikely that the result is reproducible to a point enough for medical diagnostic purposes.

Labeling and Detection

The current labeling technique for fluorescent labeling, which requires coupling of a bulky fluorescent dyes (fluorochromes) to an amino acid or a nucleotide in the molecule of interest to facilitate detection. While DNA and RNA are more forgiving in how much structural modification can be done to them before they stop binding to their complementary sequences, proteins are not. A fluorochrome can be five times as big as an amino acid, thus such modification can significantly alter the folding and three dimensional structures of proteins. In addition, the modification itself can destroy binding regions recognized by binding agents. For example, Cydye that modifies Lysines in a protein will effectively change all the epitopes that contain Lysine. As a result antibodies that normally bind to these epitopes will no longer bind.

One major disadvantage of the current labeling techniques is that the labeling reaction has to be performed at the time of analysis. Therefore labeling reactions can vary significantly between different operators. In addition, a quick method for labeling is necessary to have any commercial value. Most labeling techniques that preserve the delicate natures of proteins require longer processing time. One improvement that can significantly change the process is to allow samples to be prelabeled and validated prior to analysis.

Radioactive labeling solves the problem by making the labeling groups much smaller, or better yet, by direct incorporation of labeled amino acid yielding label with no chemical modification. Such metabolic labeling techniques are done routinely in research laboratories. Once labeled, the common detection technique is exposing to X-ray film that record the radiation. Since X-ray film has a limited linear range, thus usually not suitable for quantitative analysis, other methods have evolved over time. A commonly used method comprises the use of a phosphorescent storage imaging screen. The high-energy radiation excites the storage material's electrons into their phosphoresced state at which they will remain until excited again by the right quanta of energy. Using that principle, a phosphorescent screen captures some of the radiation energy, stores it, and will emit radiation when read with a tuned laser. Such devices are also commonly used in photography.

Yet another method that enables quantitative analysis is Scintillation Counting. A fluorescent material is mix with the radioactive sample so that when the material is strike with high-energy radiation it will give up light for easy detection and quantification.

Radioactive labeling is also much more sensitive than any other non-radioactive labeling methods. As a result, this method can also be used to replace existing working methods when higher sensitivity is required.

### SUMMARY OF THE INVENTION

Briefly stated, this invention provides a novel means of detection and quantification to enable comparison of molecules from two or more samples on arrays of binding agents. The novel method allows competitive bindings of two samples on the same array making quantitative comparison possible. In addition, improved methods uses radioactive labeling offers the highest sensitivity of all available detection methods.

The present invention discloses a method of analysis comprising the steps of: (1) labeling two samples of test molecules, each with a unique labeling agent; (2) mixing the samples of test molecules into a homogenous mixture of samples of test molecules; (3) applying the mixture to an array of binding agents; (4) rinsing away any un-reacted or unbound test molecules from the surface of the array; and (5) analyzing the surface for any indication of reaction between resident molecules and test molecules.

A variation of the invention discloses a method of using one readily detectable label (of any kind of labeling agent or distinguishing characteristic of a molecule) as a standard to perform the analysis. The other sample to be multiplexed with contains a dummy label for the purpose of making molecules in both samples behave the same way. For example, naturally occurring carbon-12 would be considered a dummy label for radioactive carbon-14. The method of analysis comprises the steps of: (1) mixing quantities of a standard with two different quantities of samples to produce mixtures having two or more different proportions; (2) applying the different mixtures to identical arrays; (3) washing away unbound molecules; (4) reading the signal from each array; and (5) calculating the amount of various molecules present in the tested sample relative to the amount in the standard sample using the signal variation between various mixtures.

A major advancement in labeling and detection of multiple samples in this invention is the use of radioactive isotopes. Unlike other labeling and bioconjugation techniques, radioactive labeling does not require putting large tags onto biological molecules that often result in significant physical and chemical changes in the properties of the biological molecules. Radioactive labeling can also be done by metabolically adding the isotope to live cells as they synthesize the molecules of interest. The resulting molecules thus have the exact identical chemical structures, functions, and interactions with other molecules. This method allows multiplexing by using different radioactive isotopes such as ³H, ¹⁴C, ³⁵S, ³³P, ³²P . . . etc. and differentiating between the isotopes' combined signal from the mixture using their difference in radiation energy or half-life.

An object of this invention is to provide a method for profiling and comparing samples on the array by multiplexing them on the same array. The method thus helps speed up discovery of biomarkers that are relevant to disease conditions or drug efficacy.

Another object of the invention is to provide a much more sensitive method for labeling, detection and quantitative comparison using radioactive labels.

Another object of the invention is to provide a method that only requires one labeled standard to be used to detect or quantify the amount of various molecules in other unlabeled samples. A radioactively labeled molecules known to bind specifically to a binding agent can be used to detect similar molecules but are not labeled by competitive binding to the binding agent. A further object of this improved version is to provide a medical diagnostic tool and a proteomic-based tool for clinical research. Such a tool can also be used to perform clinical lab testing in point-of-care diagnostic instrument using lab-on-a-chip concept.

A further object of this invention is to provide an improved method for proteomic, genomic, transcriptomic, metabolomic.. .etc. analysis using radioactive isotopes of different radiant energy level or different half-life to enable simultaneous detection and quantitative comparison of multiple samples of proteins, DNA, RNA, sugars, fatty acids, and other molecules without rendering the molecules incomparable or unsuitable for analysis purposes, e.g., chemically modifying them.

A further object of this invention is to teach a method of preparing labeled samples for performing analyses without altering the chemical structures of molecules within. The method uses neutron bombardment to make regular isotopes radioactive or just heavier than normal isotopes. Such labeling enables these samples to be used as standards to be quantitatively compared with other unlabeled samples.

### BRIEF DESCRIPTION OF THE DRAWINGS

FIG. 1 is a flow chart describing the process of comparing molecules' or biomarkers' abundance between two samples.

### DETAILED DESCRIPTION OF THE INVENTION

The invention provides a method of comparing profiles of a plurality of molecules such as proteins derived from comparable sources on an array. The molecules are captured and displayed by binding agents immobilized on the array so that detection and quantitative analysis can be performed. A typical antibody array would contain a specific library of monoclonal antibodies having unique specificity affixed to the solid support. Antigens from different samples were tagged or labeled with different labeling agents for detection differentiation and comparison after they are bound to antibodies. The objective is usually to profile the binding of monoclonal antibodies to any labeled target antigens from a first source, a second source, a third source, and so on. The source of antigen can be selected from the group consisting of a cell, cell lysate, tissue, body fluid, and any part or whole of an organism.

The purpose of profiling and comparing antigens is to differentially display the antigens whose levels of abundance vary due to events such as drug treatments, disease conditions, or just abnormal physiological conditions. Comparative results can give insights into disease biomarker discovery which can later be used for disease diagnostics. Additionally, the mechanism of action of a drug can be better understood so that future drugs can be more easily identified. A typical profiling experiment can use one source comprising of antigens from a known disease condition, i.e. samples taken from cancer patient while the other source comprising of antigens from a normal/healthy patient. The changes in any amount of proteins, RNA, metabolites . . . etc. in blood, specific tissues, or cell types can later become biomarkers for use in detecting that particular type of cancer in other patients who exhibit the same changes.

Alternatively, a first source may comprise antigens from a known drug-treated subject, while the second source may comprise antigens from a non-treated subject. Once the biomarkers that change due to drug treatments are identified, these same biomarkers can be used to screen potential compounds for therapeutic activity using the same setup.

The Making of an Array

The preferred device essentially comprises a pattern or array of minute antibody-coated spots on the surface of a support. Each antibody spot or group of spots is made up of antibodies of a different specificity. The spots serve as tiny, specific immuno-adsorbents for antigens. The presence of any antigen by cells may be detected and its relative amount between samples compared by determining to which antibody-coated spot the antigens bind and the relative quantity of labels detected. A large number of different antibody-coated spots can be assembled on a very small portion of the surface of the support. In addition to antibodies, the array can also have other types of binding agents preferably with high specificity.

A solid support suitable for use in the method of the invention includes a support made of all or any materials on which a binding agent can be immobilized for making a matrix of different binding agents for screening. Thus, natural or synthetic or chemically modified or unmodified materials can be used as the solid substrate, for example glass, metal, plastic, polymer, membrane, nylon, nitrocellulose, polyvinyl diflouride, and paper, polysaccharides, such as cellulose based materials for example, cellulose derivatives acetate and nitrocellulose, dextran, polymers such as, polyethylenes, polystyrenes, unsaturated carboxylic acid esters, vinylidene chloride, dienes, or compounds with nitrile groups such as acetonitrile, vinyl chloride/propylene, or vinyl chloride/acetate copolymers, natural fibers such as cotton and synthetic fibers such as nylon, inorganic materials such as silica, glass quartz, or ceramics, latexes, such as colloidal aqueous dispersions of any water-insoluble polymer, magnetic particles, metal derivatives, and other materials capable of acting as a solid support for this invention. The solid support can be in the form of a microtiter plate, a chip, a sheet, a cone, a tube, pellets, particles of some such similar configuration of the materials selected for use. Additionally, the solid support can take the shape of a rectangular wafer, or some other such easily manipulable shape which can be adapted to use by a robot in highly mechanized screening procedures. The shape of the solid support should lend itself to fixing the binding agents onto it, and also should provide a grid or location system for identifying the place on the support that an antigen binds when such binding occurs as well as facilitates the detection process.

To form the array, a plurality of binding agents such as monoclonal antibodies, aptamers, adsorbance materials are placed (i.e. adsorbed, affixed or immobilized) onto the solid support by a commercially available arrayer. The antibodies are fixed onto the solid support in a manner that the binding portions of the antibodies or fragments are accessible to the antigens or to the agents that are made to contact the solid support. Thus, generally, a non-binding region of the monoclonal antibody or fragment is used to affix the monoclonal antibody to the solid support. Upon hybridization with an antigen, the antibody-antigen location can be accurately identified.

A simple version of the above array can be made by spotting monoclonal antibodies onto a PVDF membrane. The hydrophobicity of the membrane allows the membrane to retain these antibodies (by adsorption). The rest of the membrane is then saturated with a protein source that is not likely to interfere with the use of this membrane for analysis such as non-fat milk or bovine serum albumin.

In addition to binding agents spotted to form an array, special arrays will also have radionuclides, or tags such as biotin spotted either by themselves of as modified part of a molecule. These special tags will serve as a standard by which two or more arrays can then be compared. The use of this type of array will become clear when competitive multiplexing method is described further in the preferred embodiments.

Molecules derived from a sample may be processed to some extent, depending on practicality, before being used in molecular analysis such as an array analysis. One skilled in the art will be able to determine the practical limit of processing without undue experimentation. Different methods of processing may entail detergent lysis, fractionation as commonly understood and practiced as ion-exchange chromatography, salt-cut precipitation, and size-exclusion chromatography.

Another means of labeling a particular type of molecule is to capitalize on a natural and distinguishable characteristic of a molecule that can be observed and used to identify and quantify that particular type of molecule. To capitalize on a distinguishing characteristic, it is helpful if the distinguishing feature is not altered in the mixing and array processes. If the distinguishing characteristic is reliable and sustainable, a chemical label, tag, or biomarker is not necessary to successfully label a molecule with a “labeling agent”. Thus, for purposes discussed herein and concerning the present invention, a “labeling agent” includes a chemical tag or label that is introduced to a molecule, or, it may be a distinguishing characteristic already a part of the molecule. Clearly then, the unique labeling agent may either be incorporated into molecules in the sample during biosynthesis or be put on as a tag by chemical modification, or simply be a natural molecular feature that is assigned as a label for purposes of the array and analysis.

The quantities of molecules in these samples are then more easily estimated by the quantity of label. Equal (or comparable) volumes and concentrations of the negative and positive controls are mixed together with the sample to be tested (in multi-labels methods). Once mixed, the sample fractions should be re-suspended in the appropriate buffer condition and incubated for enough time to reach homogeneity.

Spotting

The antibodies and binding agents can be arrayed on a microarray slides similar to the microarray slides manufactured by Perkin Elmer. The slides are coated with adsorbance materials or activated surface chemistry to bind or covalently link to binding agents. Of particular use is the method used by Khrapko, et al (1996) for immobilization of oligonucleotides, which can be modified for protein coupling, or Versalinx (Prolinx, Bothell, Wash., USA) slides.

Alternatively, one can also couple the antibody through its SH groups to acrylamide that has been modified to include SH groups. The Versalinx slides are pre-modified with salicylhroxamic acid. The phage can be easily modified with phenyl boronic acid and a complex between the pehylboronic acid and salicylhorxamic acid is formed to immobilize the antibody on the glass slide. Alternatively, the slides are coated with resins that impart a positive charge to the slides, which are commercially available. The phage or the bacteria can be arrayed directly on these slides using an automated slide spotter.

Yet another way to immobilize the antibody is to engineer the cDNA that codes for 6-Histidine moieties contiguously with the amino terminal or the carboxyl terminal of the antibody or the Fab fragment. Stomolysin or similar protease sites are engineered on either end of the antibody-Histidine tag fusion protein for easy excision of the antibody such that the antibody or Fab fragment will carry the Histidine tag. The Histidine residues can be utilized to bind the antibody or Fab to slides which have nickel immobilized on their surfaces. Published procedures for immobilizing nickel to glass slides or other supports are available.

The array should now constitute a substrate along with the resident molecules and any molecules derived from the positive and negative samples that have reacted with the resident molecules. The array may now be observed for the presence of the different and unique labels associated with the positive and negative samples, respectively.

Eventually, the array may be stripped of the homogenate mixture and used again or may be disposed, depending on the particular needs of the analyst.

### DESCRIPTION OF THE PREFERRED EMBODIMENTS

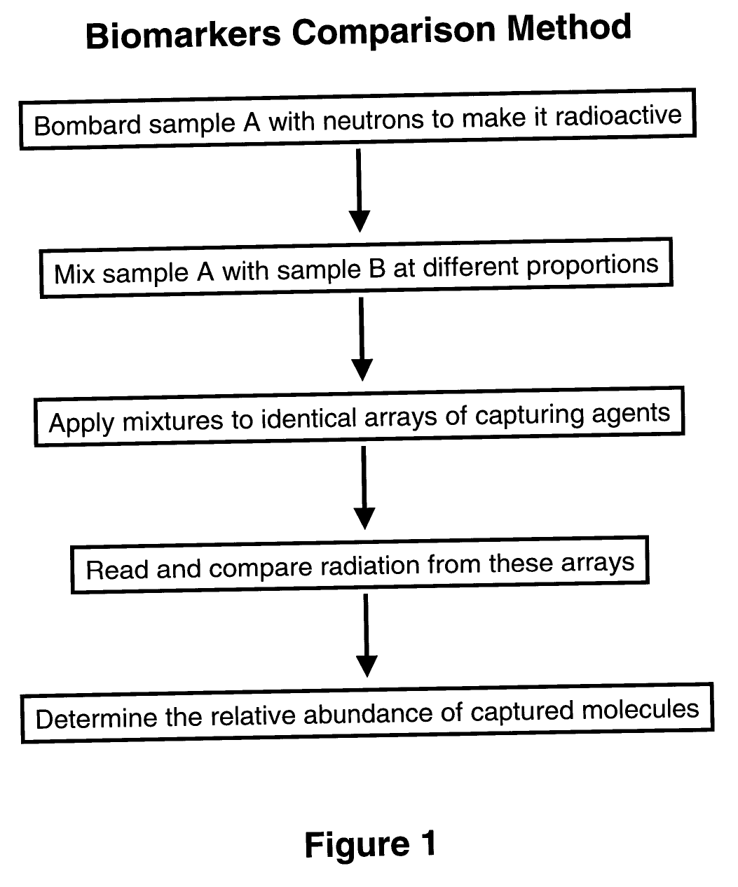

With reference to FIG. 1, a first preferred embodiment of the invention demonstrates how differences in level of abundance of many molecules from two samples can be analyzed and compared when one is labeled. The preferred labeling method use neutron bombardment to add neutrons to existing atoms in test molecules within the samples. The additional neutrons make the molecules heavier or even radioactive. Alternatively, the labels especially radioactive isotopes can be incorporated as natural parts of the molecules in the case of live cells actively synthesizing the molecules of interest. Natural incorporation can also be done by replacement of existing atoms with radioactive isotopes such as replacing hydrogen with tritium by desaturation and resaturation. Once labeling is completed, the samples are mixed to homogeneity and applied to arrays for analysis. The binding agents on these arrays bind to and profile different types of molecules at distinct locations on the arrays. The radiation from these locations is detected and quantified. When a labeled sample is mixed with unlabeled sample at different proportions and then applied to identical arrays the resulting amount of radiation signal can be used to interpret the relative amount of similar molecules in unlabeled sample relative to those in labeled sample. In addition, when captured molecules are required to be identified by mass spectrometry, the origin of molecules from either sample can also be differentiated by their slight difference in mass. Importantly, the method described provides a way for molecules having varying abundances to be discovered.

A second preferred embodiment uses other labeling methods including bioconjugation or covalent modification of molecules to add label or tags onto the molecule of interest. One sample can be tagged with a distinguishable label, tested, and then uses as standard. The standard is then mixed with sample to be tested (unknown) at two or more different proportions and then applied to two identical arrays. The signals from both arrays are read and used to calculate the ratio of unlabeled vs. labeled molecules for every particular spot. Because tagged molecules in the standard can behave differently than untagged molecules in the sample, the standard needs to be validated. This is done using similar experimental scheme as described in the first preferred embodiment where neutron bombarded sample will be compared against tagged sample. Both of these samples are of the same origin for the purpose of validating that the bindings of certain molecules have not been affected.

While the preferred labels for this embodiment are radioactive isotopes, it is not constraint to just radioactive isotopes. The molecules can be labeled with small tags, such as biotin to be detected with strepavidin or avidin couple to horse radish peroxidase or alkaline phosphatase to generate an enzymatic reaction with detectable end points. Additionally, the molecules can be labeled with big fluorophors if the dye can be incorporated into these molecules in such a way that it won't interfere with the essential properties of the molecules for the particular assays (as determined later on by validation assay). Because the labeling process does not have to be done during the time of analysis, the reagent can be prepared and validated commercially in advance to meet certain standards. If the reagent used as a standard is labeled with non-radioactive tags, then it can be tested and validated first using radioactive labeled molecules as a standard.

A third preferred embodiment covers the use of other molecules besides binding agents on an array. These molecules are spotted on for the purpose of standardizing and comparing across similar arrays. For isotope labeling, same amounts of various isotopes are spotted on the array acting not only as a standard across arrays but also as a calibration tool for calculating screening of a particular type of isotope. For biotinylation labeling, a fixed amount of biotins are spotted onto each array to enable comparison and normalization between two or more arrays.

A fourth preferred embodiment comprises alternate methods to label biological samples without altering their chemical structures or property. Biological samples, such as those from humans, can not be readily labeled with radioactive isotopes by metabolic incorporation because such labeling would require ingestion of radioactive materials. Nonetheless, labeled human biological samples are required to enable discoveries of biomarkers in disease condition or drug treatments in human. To overcome the obstacle, this method incorporates neutron activation—the same method used to make most artificial heavy and radioactive isotopes. Letting biological samples be bombarded with neutrons from source such as nuclear reactors or other neutron generating device can turn the samples radioactive over time. Briefly, samples contain stable isotopes such as hydrogen, carbon-12, sulfur-32, phosphorus-31, and few other less abundant trace elements that can gain additional neutrons and become radioactive isotopes such as tritium, Carbon-14, Sulfur-35, Phosphorus-32 or Phosphorus-33 as a result of neutron bombardment. In addition other stable isotopes are also formed as a result of neutron bombardment. Isotopes such as ¹⁵N, and ¹⁸O can code these samples to be differentiated from regular isotope by mass spectrometry.

The above descriptions provide methodologies for which a broad spectrum of particular analysis may apply. Accordingly, the following examples are provided to further enhance the users understanding of the invention but in no way are intended to limit the particular application of the method described above.

### EXAMPLES

### Example 1

A cell line is used to study the effect of a drug and also to develop new high-throughput screenings for new drugs. Cells are grown in identical ways in various culture flasks at the beginning before separating into different groups. One group is treated with a known drug (with known therapeutic efficacy) while the cell are metabolically labeled with ¹⁴C-Cystein and Methionine. Another group is treated with vehicle control (the same amount of solvents used to deliver the drug) while the cells are metabolically labeled with ³H-Cystein and Methionine. Other groups are treated with potential compounds while being labeled with ³⁵S-Cystein and Methionine. Samples from ¹⁴C and ³H labeled cells are mixed together and apply to protein arrays to look for changes in protein expression. The proteins from both samples compete for binding to binding agents on the array. As a result, if more of a particular protein is present in one sample, then more of that protein would also bind to its specific binding agent in the exact proportion. The array is then washed with different stringency buffers to retain only certain bindings and then read for radioactivity.

Detection: Tritium emits weak beta radiation that more than 95% can be blocked completely by a thin layer of saran wrap. Carbon-14 and sulfur-35 emit stronger radiation that approximately 50% pass through the saran wrap. Using this principle, reading all of the signal from the array and then reading the signal screened by a thin film such as the saran wrap will enable one skilled in the art to calculate how much signal belongs to tritium and how much belongs to ¹⁴C and ³⁵S. In addition, radiation from individual isotopes can also be quantified with all three isotopes mixed together. This is accomplished by first reading the total signal, then reading the signal with a tritium screen. The array may be stored for a period of time before reading again. If the storage period is 87.4 days (one half-life of ³⁵S), then when it is read again, the reduction in total signal is equal to half the signal from ³⁵S. Simple mathematical calculations will quantify the signal from ¹⁴C and tritium. In addition, the arrays that are destined for these types of experiments may contain spots of these radioactive isotopes on them to enable better calibration. Specialized software, also known to one skilled in the art, ultimately can perform all of the calculations necessary to separate the combined signals between different isotopes.

After reading signals from each isotope, these signals are compared, for their respective increase or decrease in the different proteins' expressions, and new drug treatments are pinpointed. Spots on the array that show significant changes are used as biomarkers for further drug discovery. Together, these spots form a “profiling map” that one would look for in test compounds. In addition, the protein from any spot can be identified if the binding agent at that spot is known to capture a specific protein. If not, then the same antibody can be used to capture a large amount of that particular protein by methods such as immunoprecipitation, and then identify using mass spectrometry.

Using the “profiling map”, one can look for similar changes in compounds being tested. Compounds that show enough similarity can be further studied for their comparable therapeutic effect. In addition, knowing the biomarkers that change as a result of drug treatments can provide insights into how a particular drug works or what possible drug-targets there are. All of this knowledge can lead to better ways in the development and screening of new drugs.

### Example 2

Post-translational modifications play an important role in protein functions. Protein can be modified by the addition of phosphates, sugars, lipid, or steroids, methyl groups, nitrate groups . . . etc. These modifications may be studied using the same approach described in this invention for labeling samples with different isotopes and then profiling on an array.

Phosphorylation Study

Two sets of cells are plated and treated identically from the beginning, then one set is treated with drug while being labeled with ³²P phosphates, and the other set is treated with a vehicle while being labeled with ³³P phosphates. After a defined treatment time, the labeling media is washed away. The cells are next harvested and mixed together for analysis. Protein samples can be prepared for profiling on protein arrays while RNA samples can be prepared for profiling on gene arrays (a long labeling time will also label RNA and DNA). Protein-RNA and protein-DNA interactions can also be studied in addition to just protein-protein interactions.

### Example 3

B. anthracis is growth in medium with radioactive precursors and induced to make antigens normally present in human infection such as lethal factors, edema factors. The specific radioactive proteins are purified to be used as standards. Antibodies are also made against these antigens to use in diagnosing early anthrax exposure. Antibodies are immobilized on beads or array for the purpose of the assays.

The amount of standard used will always contain more antigen than available antibody on the array. Thus this standard alone or in combination with other sample will always saturate the binding capacity of the array. The radiation signal is read when the standard is used alone and as a mixture with healthy samples. Both of these readings should be approximately the same to make reliable diagnosis. The mixtures of standard and samples from anthrax exposed animals are used to determine if there are any changes in radiation reading. A change in the radiation reading means positive detection of the same antigens used in the standard and captured by antibodies on the array. Different mixtures with samples taken at different stages of exposure will determine how early the assay can be used to detect anthrax exposure.

### Example 4

Normal human clinical samples are taken and frozen for preservation. These samples are then bombarded with neutrons for neutron activation. When the samples have become sufficiently radioactive, they can be used as standards to look for changes in other samples by using the steps below as a guide:

1. Use a fixed amount of standard on the array to obtain standard only signal. The amount used must be sufficient to saturate every spot on the array. This is determined when increasing the amount used won't increase the signal read any further.

2. Mix the fixed amount of standard with approximately the same amount of sample to be tested. Determine the percentage of signal reduction at spots that capture housekeeping proteins such as βactin.

3. Use the percentage of signal reduction in other spots to determine the relative amount of proteins between samples and standard. Housekeeping proteins represent equal amount between sample and standard. If signal reduction percentage is higher at a spot (compared to percentage reduction of housekeeping proteins) then more proteins from sample than standard is present at that spot (and vice-versa). Therefore that particular protein is more abundance in the sample compared to the standard.

4. An additional mixture can be used with slightly different mixing ratio if necessary to increase the reliability of the analysis. Such additional steps would be required when validating the assay. If using 1:1 of standard:sample mixing ratio resulting in 50% signal reduction, then 1:2 mixture would result in ˜66.7% signal reduction and so on.

Using samples from normal/healthy donors bombarded with neutrons to make standards, one can look for changes of biomarkers in diseased or drug-treated patients. These biomarkers can later be used to diagnose the diseases or test drugs' efficacies.

### Example 5

Similar to example 5 this method use non-radioactively labeled sample as standard. Because such labeling necessitate the addition or chemical modification of existing molecules, the standard needs to be validated to make sure that such modification is compatible with the array used. For such validation, competitive binding with neutron activated samples is done. Clinical samples are tagged with biotin or other non-radioactive labels so that they can be readily detectable and quantifiable. The tagged samples are used to compete with the identical samples that have been neutron activated. Any spots with consistent signal reduction indicated that the biotin tags have not interfered with protein binding to those spots. Those are the spots that can be used with the biotin tagged standards. Once validated, an array containing only good working spots plus some spots with fixed amount of immobilized biotin (or other labels) can be used for quantitative analysis. Additionally, when performing multiplexing with neutron activated samples, non-radioactive labels are quantified to establish references.

### Example 6

Cells are grown in ³²P or ³³P phosphates while undergoing different treatments. Any resulting difference in RNA expression as a result of these treatments is then examined. After labeling, the cells are collected and approximately equal amounts from ³²P and ³³P labeling are mixed together for comparative analysis. RNA is extracted, and DNA is digested away using RNase-free DNase. The RNA is then bound to a DNA array for profiling and comparison. The resulting individual signal from ³²P VS. ³³P can then be calculated by selective screening or by pre-decay and post-decay comparison. Briefly, detecting total signal and partial signal blocked by an X-ray film to quantify signal from each isotope based on the different percentage of radiation from each isotope passing through the film. By decaying for a period of time and match the decaying amount to the right mixture of isotopes to quantify the amount of each isotope. This type of labeling and detection allows much higher degree of sensitivity thus only very small amount of samples are required for the analysis. Furthermore, the improved sensitivity also enables detection of extremely low abundance RNA.

### Example 7

When labeling methods discussed herein can not be incorporated due to some limit on practicality or sensitivity of a biological molecule, often, DNA or RNA interaction with proteins can be substituted as a label. Accordingly, even when an analysis is not possible with other labeling methods, the processes between DNA, RNA, and protein may provide a sufficient distinguishing characteristic to identify a molecule after mixing. This is of particular advantage because interactions between proteins, DNA, RNA, lipids . . . etc. are very important in cellular metabolism. Such interactions offer further uses in drug targeting.

In this example, the term “label” includes a distinguishing characteristic and does not require the inclusion of a chemical tag, biomarker, or labeling agent.

To assist in separating DNA and RNA samples, agarose beads may be used to immobilize one type of molecule in and an array can be used to capture the other type of molecule.

### Example 8

DNA methylation is well known as a distinguishing DNA characteristic, particularly when concerning diseases, such as cancers or other regulatory mechanisms such as genetic imprinting. Determine the extent of DNA methylation and which genes have been methylated can facilitate the diagnosis of cancers.

For DNA methylation analysis, cells samples are collected from patients to be used for comparison with a normal or healthy donor's sample. DNA is extracted, and then methylated with either tritium or ¹⁴C labeled methyl donor compounds such as Adenosyl-L-Methionine, S-[methyl-³H] or Adenosyl-L-Methionine, S-[methyl-¹⁴C]. Then equal amount of DNA from both normal control and patient's sample are mixed together for restriction enzyme digestion. The digested fragments are applied to DNA arrays and the signal from tritium and ¹⁴C detected and quantified.

The reasoning here is that any methylation site on the DNA that has already been methylated won't be methylated again with radioactively labeled methyl groups. As a result, the more DNA has been methylated, the fewer labels it will acquire in the mixing process.

In addition, multiplexing patient DNA against normal donor DNA allows identification of genes that are abnormally methylated. These genes can be used as biomarkers to diagnose or better understand the disease in the future. When the methylations of such genes are well characterized, it will be possible to only use the patient's samples alone, along with the patient's history, on a DNA array to determine the methylation of any particular gene. These genes are of special interest in contributing to the disease and may be used to devise diagnoses or treatments

Importantly, while this example describes using a radioactive methyl group to perform the experiment, those skilled in the art can also use non-radioactive labels to modify DNA and perform the study.

The examples herein should not be interpreted as an exhaustive or comprehensive list of the possible applications of the present invention. The present invention is a method for combining distinguishable molecules, exposing them to an array, and finally observing a characteristic of the distinguishable molecule after array. Analysis and observations may be automated to such an extent that clinical diagnosis is entirely instrumental and automated. In fact, form sample collection, mixing, array application, and analysis may be completely automated and not require any manual handling or processing.

Having disclosed my invention in such terms as to enable those skilled in the art to understand and practice it, and having identified the presently preferred embodiments thereof, I CLAIM:

What is claimed is:

1. A method of analysis comprising the steps of:
(a) labeling at least one sample of test molecules each with a label;
(b) mixing the labeled sample with a second sample into a homogenous mixture of test molecules;
(c) binding said homogeneous mixture to an array; and
(d) detecting at least one sample from said array.

2. The method of analysis of claim 1 further comprising the step of:
detecting and quantifying the quantity of a test molecule in at least one sample.

3. The method of analysis of claim 1 further comprising the step of:
detecting and differentiating between samples of test molecules through an observance of at least one label.

4. The method of analysis of claim 1 further comprising the step of:
comparing relative amount between samples through an observance of at least one label.

5. The method of analysis of claim 1 further comprising the step of:
automated analysis.

6. The method of analysis of claim 1 further comprising the step of:
an observational technique selected from the group consisting of: post-translational modification of protein study, protein expression study, and gene-expression analysis.

7. The method of analysis of claim 1 wherein the label is a radioactive labeling agent.

8. The method of analysis of claim 1 wherein at least one label is a distinguishing characteristic of the molecule other than an incorporated chemical tag.

9. The method of analysis of claim 1 further comprising the step of:
placing a screen over the array for selective filtering of signal from at least one sample from said array.

10. The method of analysis of claim 1 wherein a sample is labeled by neutron bombardment

11. A method of labeling a sample for analysis by neutron bombardment.

12. A method of detection using labeled molecules to detect the present of similar but unlabeled molecules by competitive binding to an array.

13. The method of claim 12 further comprises a step of quantifying said unlabeled molecules.

14. The method of claim 12 wherein the label is a radioactive label.

15. The method of claim 12 wherein said labeled molecules are prepared by neutron bombardment.

16. The method of analysis of claim 1 wherein said array comprising immobilized label.

17. An array comprising immobilized label.

18. Said array of claim 17 wherein said label includes radioactive isotope.

19. Said array of claim 17 wherein said label includes biotin.

20. Said array of claim 17 wherein said label includes fluorescence.
